(19) United States
(12) Patent Application Publication
(10) Pub. No.: US 2002/0137779 A1
(43) Pub. Date: Sep. 26, 2002

(54) Use of L-2-oxothiazolidine-4-carboxylic acid for the treatment of type 2 diabetes

(75) Inventors: Uh-Hyun Kim, Chollabuk-do (KR); Myung-Kwan Han, Chollabuk-do (KR); Young-Mi Shin, Chollabuk-do (KR); Kwang-Hyun Park, Chollabuk-do (KR); Kum-Jae Park, Inchon (KR)

Correspondence Address: FRANCIS J MAGUIRE, BRADFORD GREEN BUILDING FIVE, 755 MAIN STREET, P O BOX 224, MONROE, CT, 06468

(73) Assignee: Advanced Biochemicals Inc.

(21) Appl. No.: 09/875,514

(22) Filed: Jun. 6, 2001

Related U.S. Application Data
(60) Provisional application No. 60/295,294, filed on Jun. 1, 2001.

(30) Foreign Application Priority Data
Feb. 14, 2001 (KR) 2001-7366

(51) Int. Cl. A61K 31/426

(52) U.S. Cl. 514/369

(57) ABSTRACT

The present invention provides the use of L-2-oxothiazolidine-4-carboxylic acid for the treatment of type 2 diabetes(NIDDM; non-insulin dependent diabetes mellitus). The present invention also provides the pharmaceutical composition containing the chemical L-2-oxothiazolidine-4-carboxylic acid as an active agent for the treatment of type 2 diabetes and the method for the treatment of type 2 diabetes comprising administering to a mammal having the same disease.

Use of L-2-oxothiazolidine-4-carboxylic acid for the treatment of type 2 diabetes

### BACKGROUND OF THE INVENTION

1. Field of the Invention

The present invention relates to the use of L-2-oxothiazolidine-4-carboxylic acid(OTC), its derivatives, or its pharmacologically acceptable salts of the formula(I)

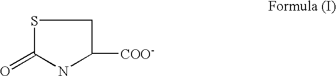

as pharmaceutical active compounds, for the treatment of type 2 diabetes(non-insulin dependent diabetes mellitus). More specifically, the present invention relates to the use of L-2-oxothiazolidine-4-carboxylic acid, its derivatives, or its pharmacologically acceptable salts of the fomuula(I) compounds as type 2 diabetes treating agents, pharmaceutical composition containing them as active compounds and a method of treating a mammal having a type 2 diabetes comprising administering to said mammal pharmaceutically effective amount of the compounds of the formula(l).

2. Discussion of the Background

Diabetes is a chronic metabolic disorder to overcome in modern medical science, and the incidence of more prevalent form, type 2 diabetes is increasing with years since it is closely related to obesity, insufficient exercise, aging, and genetic disorders, such as resistance to insulin or defect of insulin secretion(Eung-Jin Lee et al., Diabetes Science, Korea Medicine, 1I31, 1998; Jameson, J. L., Diabetes Care.. 21 (Suppl.l1): S5-S 19, 1998; Pickup, J. C. & Crook, M. A.. Diabetologia, 41: 1241-1248, 1998; Liu, K. et al., J. Med. Chem., 43): 3)487-3)494, 2000). Moreover, since the population of diabetes will increase to about 230 million by the year 2010, diabetes treating agents are urgently needed(Betteridge, D. J., New Engl. J. Med., 342: 1533, 2000).

Oral anitdiabetic agents, such as sulfonylureas, metformin, troglitazone, and acarbose have been known(Eliasson, L. et al., Science 271: 813-815, 1996; McGraw-Hill Companies, U.S.A., Part 13, Chapter 334, pp. 2060-2080, 1999; McCarthy, K. J. et al., Kidney International., 58: 2341-2350, 2000). However, the drug sulfonylureas has the problem of hypoglycemia, because it secrets insulin even when insulin is not needed in the body(Holman, R. R. & Turner, R. C., In Textbook of Diabetes, Pickup, J. C., Williams, G., Eds; Blackwell Scientific Publ. London, pp 462-476, 1991). On the other hand, metformin has been known to inhibit gluconeogenesis in hepatocytes and to increase insulin receptors, however, it is also known to cause stomach-malfunction and toxicity which can cause death by excessive dose of administration to a patient for a prolonged time(Innerfield, R. J., New Engl. J. Med., 334: 1611-1613, 1996). Troglitazone is known to increase insulin sensitivity in muscle, however, it also has side effects, such as anemia, malfunction of stomach and hepatic toxicity(Eung-Jin Lee et al., Diabetes Science, Korea Medicine, 345-359, 1998; Ishii, S., et al., Diabetes, 45: (Suppl. 2), 141A(abstracts), 1996; Watking, P. B., et al., N. Engl. J. Med. 338: 916-917, 1998). Thus, the development of a drug which does not cause hypoglycemia with a minimal side effect is needed.

Surprisingly, the present inventors found that the compound of formula (I) can be used as an effective antidiabetic agent which does not have the above-mentioned drawbacks.

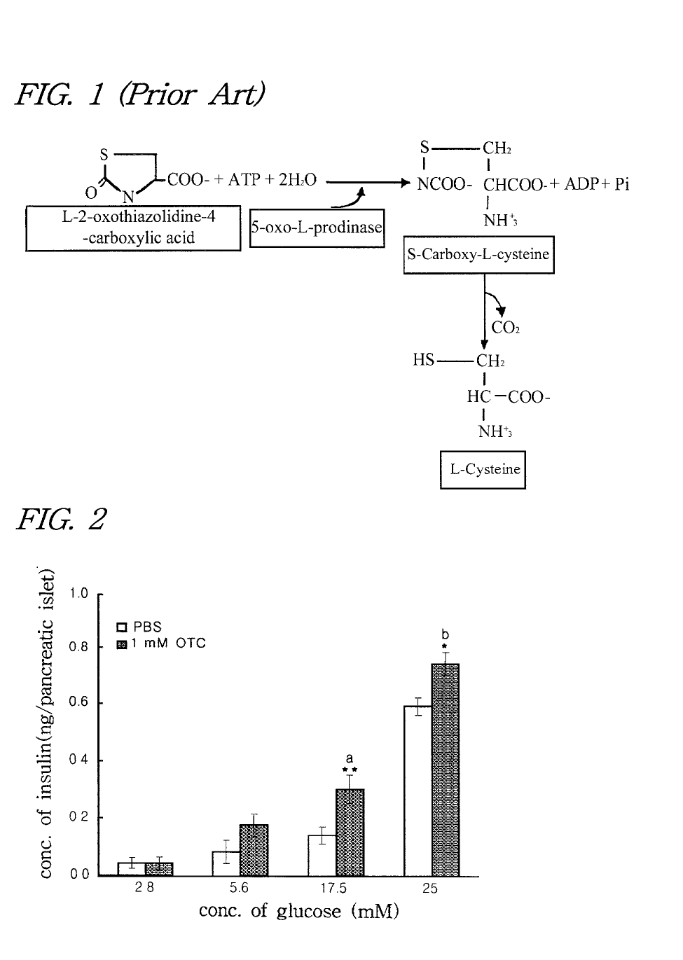

U.S. Pat. No. 4,647,571 to Meister et al. discloses that L-2-oxothiazolidine-4-carboxylic acid is cleaved into L-cysteine and C02 in the presence of adenosine triphosphate (ATP) by the action of 5-oxo-L-prolinase in a body as shown in FIG. 1. See also Willamson, J. M. & Meister, A., Proc. Natl. Acad. Sci. USA, 78: 936-939, 1981; Ensunsa, et al., Am. J. physiol., 264: E782-E789, 1993; Enderson, M. E. & Meister, A., Methods Enzymol., 143: 313-325, 1987. 5-oxo-L-prolinase is widely distributed in organisms ranging from microorganism to mammalian cells, and in many organs of mammals, especially kidney and heart. And L-2-oxothiazolidine-4-carboxylic acid has been found to be a good substrate for 5-oxo-L-prolinase(Willamson, J. M & meister, A., Proc Natl. Acad. Sci. USA, 78: 936-939, 1981; Vita, J. A. et al., J. Clin. Invest., 110: 1408-1414, 1998; Enderson, M. E. & Miester, A. Methods Enzymol., 143: 313-325, 1987). L-2-oxothiazolidine-4-carboxylic acid contains -SH group in its structure, has the effect of detoxifying the toxicity caused by free oxygen radical generated in a body(Porta, et al., J. Pharmacol. Exp. Ther., 257: 331-334, 1990; Boesgaard S, et al., Circulation, 87: 547-553, 1993), and is also known to remove endotoxin by increasing the production of L-cysteine and glutathion(Hazelton, et al., J. Pharmacol. Exp. Ther., 237: 341-349, 1985; Olney, J. W., et al., Science, 248: 596-599, 1990) and to have a good detoxifying action against the toxicity by chemotherapeutical agents, such as cisplatin, doxorubicin or cyclophosphamide(Willamson, J. M & Meister, A. Proc. Natl. Acad. Sci. USA, 78: 936-939, 1981; del Olmo, et al., Melanoma Res., 10: 103-112, 2000). It has also been known to have an excellent antidotal effect against an acetaminophen-induced hepatotoxicity. Oral administration of acetaminophen at a dose of 9.5 mmol/kg in mice resulted in more than 90% of mortality, however, when pre-treated with L-2-oxothiazolidine-4-carboxylic acid, the mortality rate of the animal was less than 10%(Hazelton, et al., J. Pharmacol. Exp. Ther., 237: 341-349, 1986).

Moreover, L-2-oxothiazolidine-4-carboxylic acid has been known to be a good treatment of cardiovascular diseases, such as arteriosclerosis(Vite, et al., J. Clin. Invest., 101: 1408-1414, 1998; Poon, et al., Am. J. Respir. Crit. Care Med., 158: 1109-1113) and also an excellent antidote for the toxicity due to cysteine and glutathion deficiency shown in AIDS(acquired immunodeficiency syndrome)(Roederer, et al., Proc. Natl. Acad. Sci., USA, 87: 4884-4888, 1990).

As explained in the above, L-2-oxothiazolidine-4-carboxylic acid has been known to have a good detoxifying action in the body, and known to be a drug having a good safety record which can be administered to mammals orally, intraperitoneally or subcutaneously in a dose of 2.5-15 mmol/kg(0.37-2.21 g/kg)(Enderson, M. E. & Meister, A., Methods Enzymol., 143: 313-325, 1987).

However, the use of L-2-oxothiazolidine-4-carboxylic acid, its derivatives, or its pharmaceutically acceptable salts for treatment of type 2 diabetes has not yet been reported. The present inventors found that the compound of the formula(I), its derivatives, or its pharmacologically acceptable salts can be used as antidiabetic agent based on the present inventors' finding that a composition containing the compound as an active agent can improve the insulin secretion of pancreatic β-islet cells isolated from mice, and the composition also lowers the blood glucose levels and improves the ability of insulin secretion. when administered to C57BL/KsJ-db/db Jcl which is known as a model mice of type 2 diabetes.

### SUMMARY OF THE INVENTION

Thus, the object of the present invention is to provide a new use of the L-2-oxothiazolidine-4-carboxylic acid, its derivatives, or its pharmaceutically acceptable salts for the treatment of type 2 diabetes.

Another object of the present invention is to provide a new composition containing the compound L-2-oxothiazolidine-4-carboxylic acid, its derivatives, or its pharmaceutically acceptable salts as an active agent for the treatment of type 2 diabetes. The present invention further provides methods for treating type 2 diabetes. The method comprises administering a pharmaceutically effective amount of a compound of the formula(I) to a mammal in need.

According to the present inventors' studies based on the data obtained from experiments, the major anti-diabetic action of L-2-oxothiazolidine-4-carboxylic acid(OTC) may be attributable to its insulinotropic effect which reduces blood glucose levels, although the present inventors cannot exclude a possibility that the anti-oxidant might exert its beneficial effect directly on target tissues as well as pancreatic islet cells. Therefore, OTC can be used to treat a type 2 diabetes by improving insulin secretion and lowering blood glucose levels. Moreover, since OTC functions as an anti-oxidant as described above, it can be also used for the prevention of diabetes complications, such as retinopathy, nephropathy and neuropathy.

The present inventors performed experiments to find the ability of blood glucose lowering effect and insulin secretion improving the effect of the composition containing L-2-oxothiazolidine-4-carboxylic acid as an active agent, as follows: Acinar cells were removed by washing pancreatic islets obtained from C57BL mice(weight: 25 g) by the method of Lacy P. E. and Kotianovsky M.(Diabetes, 16: 35-39, 1967) with HBSS(Hank's balance salt solution). Pancreatic islets of similar size were isolated under dissecting microscope, treated with L-2-oxothiazolidine-4-carboxylic acid(Sigma, USA) and glucose(Sigma, USA) together, or L-2-oxothiazolidine-4-carboxylic acid alone, and the concentration of insulin was measured by radioimmunoassay using insulin assay kit(Diagnostic Products Co, USA; Amersham Pharmacia Biotech Co, USA). To determine the effect of L-2-oxothiazolidine-4-carboxylic acid to affect blood glucose levels and the ability of insulin secretion in a body of C57BL/KsJ-db/db Jcl(Clea, Japan), a known type 2 diabetic model mouse, the composition containing the compound was orally administered once for 1-4 weeks. The result of glucose tolerance test and of insulin concentration by radioimmunoassay as described above showed that the drug lowers blood glucose levels and improves insulin secretion.

The compound L-2-oxothiazolidine-4-carboxylic acid, its derivatives or its pharmaceutically acceptable salts may be administered by a variety of routes. They are effective if administered orally and the compound may also be administered parenterally (i.e. subcutaneously, intravenously, intramuscularly, or intraperitoneally).

Pharmaceutical composition can be manufactured utilizing techniques known in the art. Typically, a therapeutic amount of the compound will be admixed with a pharmaceutically acceptable excipient, carrier or diluent.

For oral administration, the compound can be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In another embodiment, the compound of formula(I), its derivatives, or its pharmaceutically acceptable salts can be tabulated with conventional tablet bases such as lactose, sucrose, and cornstarch, in combination with binders, such as acacia, cornstarch, or gelatin, disintegrating agents such as potato starch or alginic acid, and a lubricant such as stearic acid or magnesium stearate. Liquid preparations are prepared by dissolving the active ingredient in an aqueous or nonaqueous pharmaceutically acceptable solvent which may also contain suspending agents, sweetening agents, flavoring agents, and preservative agents, as known in the art.

For parenteral administration, the compound may be dissolved in a physiologically acceptable pharmaceutical carrier and administered as either a solution or a suspension. Illustrative of suitable pharmaceutical carriers are water, saline, dextrose solution, fructose solutions, ethanol, or oils of animal, vegetative, or synthetic origin. The pharmaceutical carrier may also contain preservatives, buffers, etc., as known in the art.

The compound can also be administered topically. This can be accomplished by simply preparing a solution of the compound to be administered, preferably using a solvent known to promote transdermal absorption such as ethanol or dimethyl sulfoxide (DMSO) with or without other excipient. Preferably topical administration will be accomplished using a patch either of the reservoir and porous membrane type or of a solid matrix variety.

Even though the present inventors performed experiments hereinafter using L-2-oxothiazolidine-4-carboxylic acid and its sodium salt, one skilled in the art would easily understand that its pharmacologically acceptable salts, for example, potassium, calcium and magnesium salts, and its derivatives, such as its ester form, can also exert the same or similar effect on type 2 diabetes. Therefore, the salts of L-2-oxothiazolidine-4-carboxylic acid and its derivatives also fall within the scope of the present invention.

The dosage range at which these compounds exhibit the treating effect on type 2 diabetes can vary widely depending upon the severity of the patient's disease, the patient, the particular compound being administered, the route of administration, and the presence of other underlying disease states within the patient, etc. Typically, the compounds exhibit their therapeutic effect at a dosage range of from about 0.1 mg/kg/day to about 1,000 mg/kg/day, preferably about 1 mg/kg/day to about 500 mg/kg/day. Repetitive daily administration may be desirable and will vary according to the conditions listed above.

According to the present inventors' study, the ED50 was 15 mg/kg. ED50 is defined as 50% of maximum effective dose calculated by linear regression method. The term “treat” refers to the ability of the compounds to either relieve, alleviate, or slow the progression of the patient's disease.

### DETAILED DESCRIPTION OF THE INVENTION

Hereinafter, an outline of the experimental method performed by the present inventors is described. As can be seen, the method is composed of six steps, as follows:

First step ; After isolating pancreatic islets from a C57BL mouse according to Lacy and Kotianovsky(Diabetes, 16, 35-39, 1967), the islets were incubated for 60 min. at 37_{°C}. with 250 ^{μ-}2.0 mM of L-2-oxothiazolidine-4-carboxylic acid alone or together with 2.8-25 mM of glucose, and insulin from the supernatant was measured by radioimmunoassay,

Second step ; Determining optimum L-2-oxothiazolidine-4-carboxylic acid concentration which is appropriate for enhancing insulin secretion and lowering blood glucose levels in a type 2 diabetes mouse, by administering 0-400 mg/kg of L-2-oxothiazolidine-4-carboxylic acid orally,

Third step ; Determining the abilities of lowering blood glucose level and increasing insulin secretion ability following glucose loading, by administering a single dose of 8-80 mg/kg of L-2-oxothiazolidine-4-carboxylic acid to a type 2 diabetes mouse, Fourth step; Examining the effect of long term administration of L-2-oxothiazolidine-4-carboxylic acid on lowering blood glucose levels,

Fifth step Determining the ability of insulin secretion after oral administration of L-2-oxothiazolidine-4-carboxylic acid for 4 weeks to a 7 week old type 2 diabetes mouse, and

Sixth step: Identifying the ability of insulin secretion of the control group and the type 2 diabetes mouse to which 80 mg/kg of L-2-oxothiazolidine-4-carboxylic acid for 4 weeks determined by immunohistochemical staining with anti-insulin antibody.

The C57BL mice used in the present invention were purchased from Samtako(Daejeon, Korea), and the type 2 C57BL/KsJ-db/db Jcl mice were purchased from Clea (Japan). The chemical L-2-oxothiazolidine-4-carboxylic acid and glucose were purchased from Sigma.

The insulin assay kit was purchased from Diagnostic Product Co.(USA; Amersham Pharmacia Biotech Co.).

The chemical L-2-oxothiazolidine-4-carboxylic acid was orally administered to diabetes mice(C57BL/KsJ-db/db Jcl) with a single dose of 3.0-400 mg/kg once in the state of empty stomach or 40-80 mg/kg twice a day for 1-4 weeks continuously.

Additional features and advantages of the present invention are described in, and will be apparent from the Detailed Description of the Invention, these examples are provided solely for purposes of illustration and are not intended to be limitative.

### EXAMPLE 1

Isolation of pancreatic islets from a mouse and the measurement of the insulin secretion ability according to the administration of L-2-oxothiazolidine-4-carboxylic acid

The isolation of pancreatic islets from a C57BL mouse(weight; 20 g, Samtako, Daejeon, Korea) was performed according to the method of Lacy and Kotianovsky et al.(Diabetes, 16; 35-39, 1967). The pancreas of ether-anesthetized mouse was distended by infusion with Hank's balanced salt solution(HBSS, GIBCO, USA) containing 1 mg/ml of collagenase (Sigma, USA) via common bile duct and isolated. The isolated pancreas was digested for 20 min at 37 C while stirring homogeneously. After removing acinar cells by washing with HBSS, pancreatic islets of similar size were collected under dissecting microscope. 2.8-25 mM of glucose(Sigma, USA) alone or together with 250 ^{μM-}2.0 ml of L-2-oxothiazolidine-4-carboxylic acid were administered to the isolated 10 pancreatic islets in 100 ^{μl of Krebs Ringer bicarbonate(Sigma, USA) buffer solution containing} 10 mM of Hepes, the islets were incubated for 60 min. at 37^{°C.}, and insulin in the supernatant, from which the pancreatic β-islet cells was removed, was measured. The measurement of insulin concentration was performed by radioimmunoassay using insulin assay kit(Diagnostic Product Co., USA).

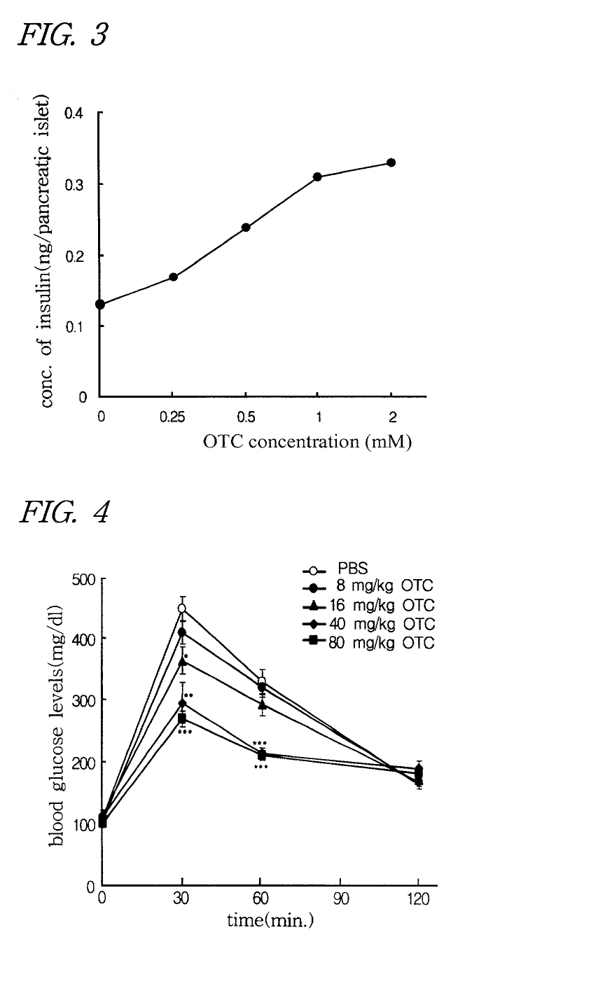

FIG. 2 and FIG. 3 show the results of the insulin secretion ability of the pancreatic 8 -islet cells isolated from the mice to which 250 ^{μ}M - 2.0 mM of L-2-oxothiazolidine-4-carboxylic acid was administered in the presence of 2.8 mM and 25 mM of glucose.

Here, FIG. 2 shows the effect of I mM of L-2-oxothiazolidine-4-carboxylic acid on insulin secretion of pancreatic islet cells, with varying concentrations of glucose, in which:

a; T-test wherein the control group to which 17.5 mM of glucose alone was administered and the experimental group to which I mM of L-2-oxothiazolidine-4-carboxylic acid(OTC) and 17.5 mM of glucose were administered,

( [symbol] [symbol] :p<0.005)

b; T-test wherein the control group to which 25 mM of glucose alone was administered and the experimental group to which 1 mM of L-2-oxothiazolidine-4-carboxylic acid(OTC) and 25 mM of glucose were administered, and

( [symbol] :p<0.05) obtained figures: average +S.D.

FIG. 3 shows the effect of varying concentration of L-2-oxothiazolidine-4-carboxylic acid on insulin secretion of pancreatic islet cells in the presence of 17.5 mM glucose.

As can be seen in FIG. 2, when 2.8-25 mM of glucose alone was administered to the pancreatic islet cells isolated from mice, low concentration of glucose(2.8 mM) did not induce insulin secretion, but higher concentration of glucose(25 mM) enhanced insulin secretion.

Therefore, the present inventors conducted experiments to examine the ability of OTC to enhance insulin secretion, employing OTC concentration ranging from 250 ^{μ}M to 2 mM in the presence of high glucose concentration(17.5 mM). The result showed that treatment with 500 mM−2 mM OTC in the presence of 17.5 mM glucose increased insulin secretion in a dose-dependent manner(see FIG. 3).

To summarize the above results, the presence of glucose is required for the stimulation of insulin secretion from pancreatic islets by L-2-oxothiazolidine-4-carboxylic acid(500 ^{μ}M-2 mM).

### EXAMPLE 2

Determination of an optimum concentration of L-2-oxothiazolidine-4-carboxylic acid to improve the ability of insulin secretion and to lower blood glucose levels with a single oral dose

The present example is to show an appropriate concentration of L-2-oxothiazolidine-4-carboxylic acid for the purpose of improving the ability of insulin secretion and lowering blood glucose levels, by varying the concentrations of L-2-oxothiazolidine-4-carboxylic acid in a single oral dose to type 2 diabetes mice.

The present inventors ascertained the effect of various concentrations of L-2-oxothiazolidine-4-carboxylic acid on lowering blood glucose level and insulin secretion. in which experiments enough sterilized-feed and water was given to 6 week old C57BL mice(Samtako, purchased from Clea, Japan) in the light condition of 12L: 12D for a week, bred the mice in the incubator of A.B.J Co.(Building 1 Chonbuk National University Medical School, Chonju, Korea) for a week, and no feed but water was given to the mice for 13 hours before insulin and glucose levels were measured.

0.2 ml of PBS (pH 7.4) was orally given to the control group(5 mice) using feeding needle, and 1.5-400 mg/kg of L-2-oxothiazolidine-4-carboxylic acid, dissolved in 0.2 ml of PBS and its pH was adjusted to 7.2 with 3 N NaOH(Junsei, Japan), was orally administered to experimental group(5 mice). After 5 hours of the drug administration, glucose(Sigma, USA) was injected intraperitoneally at the concentration of 1 g/kg, and incubated the mice for 30 minutes. Immediately after the mice were anesthetized with ether, the thoraces of the mice was incised to take blood, serum was isolated, and the insulin concentration of the mice-serum was measured by radioimmunoassay using rat insulin assay kit(Amersham Pharmacia Bio Co., USA).

The experimental results are shown in Table 1. Compared to the control group(830±52 pmol/l), 3-20 mg/kg administration group showed improvement of insulin secretion ability(p<0.05), 40-160 mg/kg administration group apparently shows more than 2 times (p<0.001), and over 400 mg/kg administration group showed salient improvement of insulin secretion ability(p<0.005).

The above results prove that 3-400 mg/kg administration of L-2-oxothiazolidine-4-carboxylic acid is preferable for the improvement of insulin secretion ability in type 2 diabetes mice.

Table 1

The effect of various concentrations of L-2-oxothiazolidine-4-carboxylic acid on insulin secretion in type 2 diabetes mice (C57BL/KsJ-db/db Jcl) bodies

| | Conc. of OTC(mg/kg) | | | | | | | | |
|---|---|---|---|---|---|---|---|---|---|
| Group | 0 | 1.5 | 3 | 10 | 20 | 40 | 80 | 160 | 400 |
| Conc. | 750 | 878 | 908 | 1103 | 1,205 | 1,535 | 1,750 | 1684 | 1350 |
| of | ±62 | ±53 | ±40 | ±93 | ±85 | ±105 | ±98 | ±130 | ±76 |
| insulin | | | | | | | | | |
| pmol/l | | | | | | | | | |

[note]
★: p < 0.05 (confidence interval compared to the control group(0) according to T-test)
★★ : p < 0.001 (confidence interval compared to the control group(0) according to T-test)
★★★ : p < 0.005 (confidence interval compared to the co ntrol group(0) according to T-test)

### EXAMPLE 3

Study on blood glucose level lowering and insulin secretion abilities after glucose intake to type 2 diabetes with a single oral administration of OTC In this example, the present inventors studied the effect of L-2-oxothiazolidine-4-carboxylic acid, at the concentration of 8 - 80 mg/kg of L-2-oxothiazolidine-4-carboxylic acid which was effective concentration for the insulin secretion, on blood glucose level lowering and insulin secretion ability after glucose was given to type 2 diabetes mice

Under the same breeding condition as in EXAMPLE 2, no feed but water was given to 7 week old type 2 diabetes mice (C57BL/KsJ-db/db Jel, Clea, Japan) for 13 hours. 0.2 ml of PBS was orally given to the control group(5 mice) using feeding needle, and 8-80 mg/kg of L-2-oxothiazolidine-4-carboxylic acid dissolved in 0.2 ml of PBS was orally administered to the experimental group(5 mice). After 5 hours of drug administration, 1 g/kg of glucose (Sigma, USA) was intraperitoneally injected and the mice were maintained for 30 to 120 minutes. The blood glucose levels of type 2 diabetes mice at various time points were measured by the glucose oxidase method using a glucose analyzer(One-touch Basic, Lifescan, USA), and serum insulin levels were determined using a radioimmunoassay, as described in EXAMPLE 2.

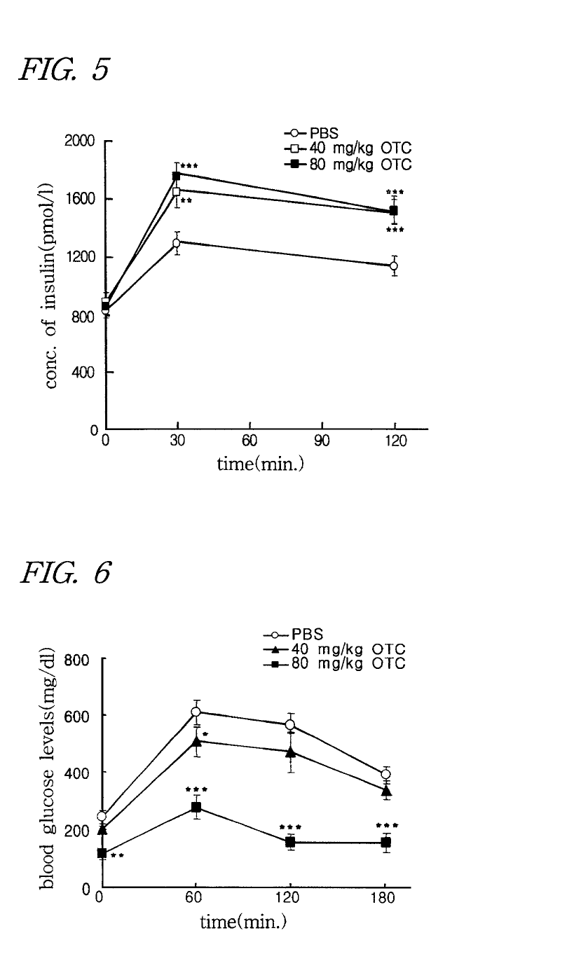

The results are shown in FIG. 4 and FIG. 5. FIG. 4 shows graphically the effect of one time a single oral administration of L-2-oxothiazolidine-4-carboxylic acid at various concentrations to 7 week old type 2 diabetes mice (C57BL/KsJ-db/db Jel) on blood glucose levels. [symbol] : p<0.01 (confidence interval compared to the control group(PBS) according to T-test) [symbol] [symbol] [symbol] : p<0.001 (confidence interval compared to the control group(PBS) according to T-test)

FIG. 5 shows graphically the effect of a single oral administration of L-2-oxothiazolidine-4-carboxylic acid at various concentrations levels to 7 week old type 2 diabetes mice (C57BL/KsJ-db/db Jcl) on blood insulin levels. [symbol] [symbol] : p<0.005 (confidence interval compared to the control group(0) according to T-test) [symbol] [symbol] [symbol] : p<0.001 (confidence interval compared to the control group(0) according to T-test)

As can be seen in FIG. 4, the experimental group to which 40-80 mg of L-2-oxothiazolidine-4-carboxylic acid per kg of mouse was administered has profound effect on the lowering of blood glucose levels. Especially, after 30 min. of glucose intake, the blood glucose level of the control group was 444.75+11.86 mg/ml. In contrast, the blood glucose levels of 40 mg/kg of OTC was 34.27% of decrease(295.00±8.50 mg/ml), and that of 80 mg/kg of OTC was decrease of 40.01% (269.20±6.85 mg/ml), respectively(p <0.001). Thus, it is obvious that these amounts of OTC have surprising effect on the lowering of blood glucose levels. The lowering effect of blood glucose levels of OTC lasted for about 60 min. after the intake of glucose(p<0.001).

As evident from the above, the oral administration of 40-80 mg/kg of L-2-oxothiazolidine-4-carboxylic acid has a distinct lowering effect of blood glucose levels in type 2 diabetes mice.

On the other hand, as can be seen in FIG. 5, the present inventors found that 40-80 mg/kg of L-2-oxothiazolidine-4-carboxylic acid showed a surprisingly prominent effect on insulin secretion by experiments in which the concentration of insulin was measured during the time period of 0-120 min. after the intake of glucose. It was apparent that the secretion of insulin was improved, since the concentration of insulin after 30 min. of the glucose intake was 1,300±78 pmol/l in the control group, however, that of insulin was 1,650±103 pmol/1(26.5% increase), p <0.005, in the case of 40 mg/kg OTC administration, and that of insulin was 1,760±94 pmol/l(35.4% increase), p <0.001 in the case of 80 mg/kg OTC administration. This improvement of insulin secretion lasted for about 120 min. after the intake of glucose compared to the control group(p <0.001).

In conclusion, when L-2-oxothiazolidine-4-carboxylic acid was orally administered in the amount of 40-80 mg/kg, the ability of insulin secretion was distinctly improved in the body of type 2 diabetes mice, and the effect lasted for about 120 min.

### EXAMPLE 4

The effect of L-2-oxothiazolidine-4-carboxylic acid administration to organs on lowering blood glucose level

The present example was to reveal the effect of lowering blood glucose levels when L-2-oxothiazolidine-4-carboxylic acid was administered to organs.

Seven week old type 2 diabetes mice were maintained as in EXAMPLE 2. 0.2 ml of PBS (pH 7.4) was given to a control group twice a day for 1-4 weeks, and 40-80 mg/kg of L-2-oxothiazolidine-4-carboxylic acid dissolved in 0.2 ml of PBS of which pH was adjusted to 7.4 with 3 N NaOH(Junsei, Japan) was orally administered to an experimental group.

The experimental results are shown in FIG. 6-8. FIG. 6 shows graphically the effect of oral administration of L-2-oxothiazolidine-4-carboxylic acid at the concentration of 40-80 mg/kg a day to 7 week old type 2 diabetes mice (C57BL/KsJ-db/db Jcl) for one week (age, 8 weeks) on blood glucose levels. z,900 : p<0.01 (confidence interval compared to the control group(PBS) according to T-test) [symbol] : p<0.001 (confidence interval compared to the control group(PBS) according to T-test)

When the present inventors orally administered 40-80 mg/kg of L-2-oxothiazolidine-4-carboxylic acid to type 2 diabetes mice for four weeks and measured the weights of the mice thereafter at one week point(age, 8 weeks), two week point(age. 9 weeks), and four week point(age, 11 weeks), the weight increased as each week went by, however, there was no weight difference.

On the other hand, there was a distinct effect of lowering blood glucose levels in the experimental group to which 40-80 mg/kg of L-2-oxothiazolidine-4-carboxylic acid was administered. More specific explanation on the effect of L-2-oxothiazolidine-4-carboxylic acid on lowering blood glucose levels will be described hereinafter.

FIG. 6 shows the results of blood glucose levels of type 2 diabetes mice to which L-2-oxothiazolidine-4-carboxylic acid was administered at the dose of 40-80 mg/kg twice a day and to which glucose was also given as explained in EXAMPLE 3, and the animal was maintained for one more week.

The present inventors measured the lowering effect of blood glucose levels of type 2 diabetes mice in the fasted state(fasted for 13 hours after the final drug administration) to which L-2-oxothiazolidine-4-carboxylic acid was given at the dose of 40-80 mg/kg a day for one week. The results were, as can be seen in FIG. 6, 242.00+23.57 mg/dl in the case of the control group, whereas, 116.00±119.74 mg/dl in the case of the group to which 80 mg/kg OTC was administered. Thus, the OTC administered group had 52.07% of the control group(p <0.01), clearly showing the lowering effect of blood glucose levels.

On the other hand, the blood glucose levels of type 2 diabetes mice, which had been fasted for 13 hours after the final drug administration and L-2-oxothiazolidine-4-carboxylic acid was given at the dose of 40-80 mg/kg a day for one week, were measured at 60 min. time intervals for 30-180 minutes. The results were, as can be seen in FIG. 6, 16.8%(507.50+52.81 mg/dl) decrease of lowering blood glucose levels in the group to which 40 mg/kg of OTC was administered, compared to the control group(610.50¹43.71 mg/dl) after 60 min. of the glucose intake(p<0.05), and 54.51%(277.50+40.89 mg/dl) decrease in the group to which 80 mg/kg of OTC was administered, after 60 min. compared to the control group(610.50±43.71 mg/dl)(p <0.01), and 72.11%(158.00±28.33 mg/dl) after 120 min. compared to the control group(566.5±40.83 mg/dl)(p<0.01). The effect lasted for about 180 minutes. Thus, one can see that OTC has an excellent lowering effect of blood glucose levels.

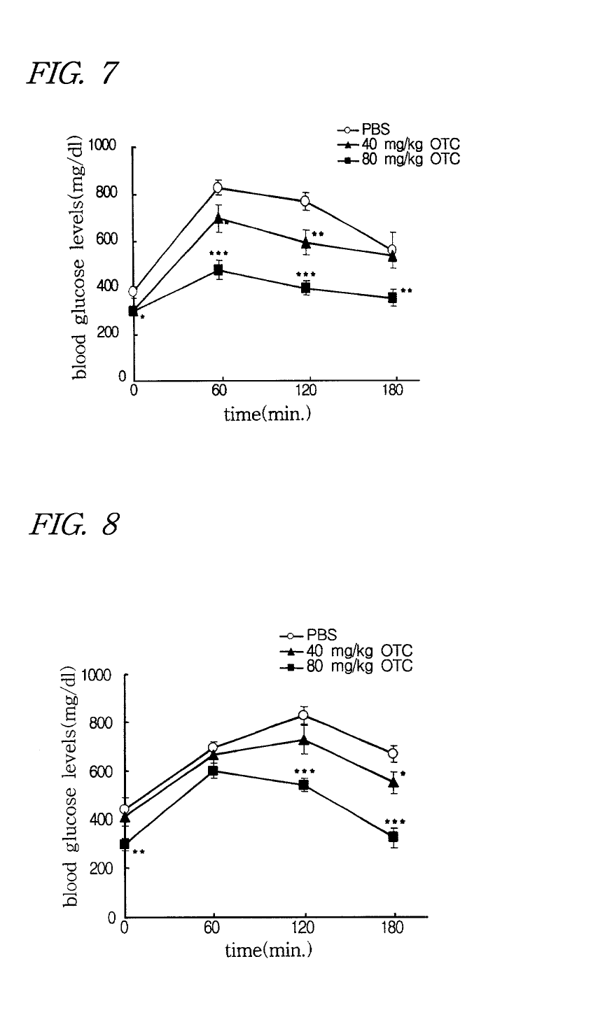

The results of blood glucose levels shown in FIG. 7 were obtained, after 40-80 mg/kg of L-2-oxothiazolidine-4-carboxylic acid was administered to type 2 diabetes mice twice a day for two weeks and after glucose was subsequently given, as described in

### EXAMPLE 3.

FIG. 7 shows graphically the effect of oral administration of L-2-oxothiazolidine-4-carboxylic acid at the concentration of 40-80 mg/kg a day to 7 week old type 2 diabetes mice (C57BL/KsJ-db/db Jcl) for two weeks (age, 9 weeks) on blood glucose levels. [symbol] : p<0.01 (confidence interval compared to the control group(0) according to T-test) [symbol] [symbol] : p<0.005 (confidence interval compared to the control group(0) according to T-test) [symbol] [symbol] [symbol] : p<0.001 (confidence interval compared to the control group(0) according to T-test)

The present inventors measured the lowering effect of blood glucose levels of type 2 diabetes mice which were fasted for 13 hours after the final drug administration and L-2-oxothiazolidine-4-carboxylic acid was given at the dose of 40-80 mg/kg a day for two weeks. The results were, as can be seen in FIG. 7, 383±8.00 mg/dl in the control group, however, 306.30±24.06 mg/dl in the group to which 40 mg/kg OTC was administered and 301.50±12.2 mg/dl in the case of 80 mg/kg of OTC administration. Thus, the OTC administered group showed 11.5% increase of lowering effect of blood glucose levels, compared to the control group(p<0.01).

At 60 min. time intervals, for 30-180 min., the present inventors measured the blood glucose levels of type 2 diabetes mice in the fasted state(fasted for 13 hours after the final drug administration) to which L-2-oxothiazolidine-4-carboxylic acid was finally given at the dose of 40-80 mg/kg a day for tow weeks. As can be seen in FIG. 7, the results showed that the group administered with 40 mg/kg of OTC had 15.83%(699.00±58.91 mg/dl) lowering effect of blood glucose levels compared to the control group(830.50±31.16 mg/dl) after 60 min. of glucose addition(p<0.01) and the group of 80 mg/kg of OTC had 42.50%(477.50±58.91 mg/dl) of the control group, thus demonstrating that OTC has a distinct lowering effect of blood glucose levels.

The group to which 40 mg/kg of OTC was administered showed 22.2%(599.50+52.46 mg/dl) lowering effect of blood glucose levels compared to the control group(p<0.05) at 120 min. after the glucose intake and the group of 80 mg/kg of OTC showed 48.81%(398.00±28.33 mg/dl) lowering effect of blood glucose levels(p<0.001). The effect lasted for about 180 min.(p<0.001).

FIG. 8 shows the results of blood glucose levels after 40-80 mg/kg of L-2-oxothiazolidine-4-carboxylic acid was administered to type 2 diabetes mice twice a day for four weeks and after glucose was given to type 2 diabetes mice, as described in

### EXAMPLE 3,

FIG. 8 shows graphically the effect of oral administration of L-2-oxothiazolidine-4-carboxylic acid at the concentration of 40-80 mg/kg a day to 7 week old type 2 diabetes mice (C57BL/KsJ-db/db Jcl) for four weeks (age, 11 weeks) on blood glucose levels. [symbol] : p<0.01 (confidence interval compared to the control group(0) according to T-test) [symbol] [symbol] :p<0.005 (confidence interval compared to the control group(0) according to T-test) [symbol] [symbol] [symbol] :p<0.001 (confidence interval compared to the control group(0) according to T-test)

The present inventors measured the lowering effect of blood glucose levels of type 2 diabetes mice in the fasted state(fasted for 13 hours after the final drug administration) to which L-2-oxothiazolidine-4-carboxylic acid was given at the dose of 40-80 mg/kg a day for four weeks. The results were, as can be seen in FIG. 8, 440.00±49.72 mg/dl in the control group, whereas, 297.00±26.51 mg/dl in the case of the group to which 80 mg/kg OTC was administered. Thus, the OTC administered group showed 32.5% more effective in lowering blood glucose levels than the control group(p<0.01).

On the other hand, the control group showed the increase of blood glucose levels(828.00±37.02 mg/dl) until 120 min. after the administration of glucose as well as in the fasted state, showing a very slow uptake of glucose and a loss of blood glucose levels.

The blood glucose levels of type 2 diabetes mice in the fasted state(fasted for 13 hours after the final drug administration), to which L-2-oxothiazolidine-4-carboxylic acid was finally given at the dose of 40-80 mg/kg a day for four weeks, were measured at 60 min. time intervals for 30-180 minutes. As shown in FIG. 8, after 180 min. of the glucose intake, there was distinct increase of lowering effect of blood glucose levels(50.50±44.21 mg/dl) in the group to which 40 mg/kg of OTC was administered, compared to the control group, and 38.8%(539±23.60 mg/dl) in the group to which 80 mg/kg of OTC was administered, after 120 min. of glucose intake compared to the control group(828.00±37.02 mg/dl)(p<0.01). The effect lasted for about 180 min.(p<0.01). Thus, one can see that OTC has an excellent effect of lowering blood glucose levels.

In conclusion, when L-2-oxothiazolidine-4-carboxylic acid was orally administered in the amount of 40-80 mg/kg for four weeks, the lowering effect of blood glucose levels was distinctly improved in the body of type 2 diabetes mice, and the effect lasted for about 180 minutes.

### EXAMPLE 5

The effect of oral administeration of L-2-oxothiazolidine-4-carboxylic acid to type 2 diabetes mice for four weeks on the induction of insulin secretion

The present example is intended to show the effect of L-2-oxothiazolidine-4-carboxylic acid, when orally administered to 7 week old type 2 diabetes mice for four weeks, on the ability of insulin secretion.

Seven week old type 2 diabetes mice were maintained as in EXAMPLE 2. 0.2 ml of PBS (pH 7.4) was given to a control group(4 mice) twice a day for four weeks, and 80 mg/kg of L-2-oxothiazolidine-4-carboxylic acid dissolved in 0.2 ml of PBS of which pH was adjusted to 7.4 with 3 N NaOH(Junsei, Japan), was orally administered to an experimental group(4 mice). Five hours after the drug was finally administered, 1 g/kg of glucose was intraperitoneally injected. Tails were cut, blood samples were taken from the tails at 60 min. time intervals until 180 min., the samples were left at room temperature for three hours, sera were collected, and the insulin concentration was measured by radioimmunoassay.

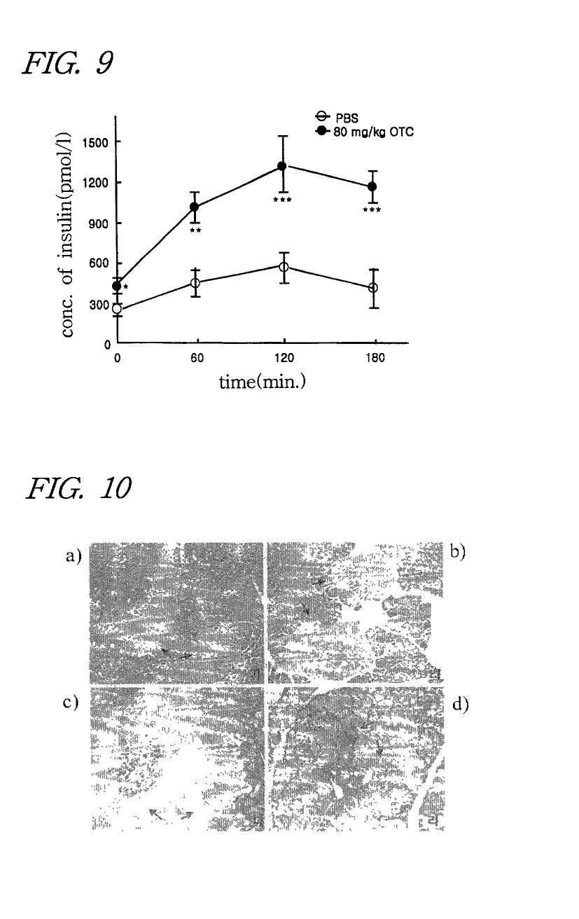

The results are shown in FIG. 9. As can be seen, the insulin concentration was 260.68±98.25 pmol/l in the fasted state and was 532.05±98.25 pmol/l even at 120 min. after glucose intake, suggesting a defect of insulin secretion. In contrast, the group to which 80 mg/kg of OTC per day for four weeks was administered showed an increase of insulin concentration compared to the control group; 450±43.50 pmol/l in the fasted state(p<0.01), and showed 4.93 times of increase(1285.04±135.60 pmol/l) after 120 min. compared to the control group, and the effect lasted for about 120 min.(p<0.001), showing an excellent effect of OTC on insulin secretion enhancement.

FIG. 9 shows graphically the effect of an oral administration of L-2-oxothiazolidine-4-carboxylic acid, given at 80 mg/kg per day for four weeks to 7 week old type 2 diabetes mice(C57BL/KsJ-db/db Jcl)(age, 11 weeks), on the concentration of blood insulin secretion. [symbol] : p<0.01 (confidence interval compared to the control group(PBS) according to T-test) [symbol] [symbol] : p <0.005 (confidence interval compared to the control group(PBS) according to T-test) [symbol] [symbol] [symbol] : p <0.001 (confidence interval compared to the control group(PBS) according to T-test)

These results further confirm the lowering effect of blood glucose levels when 80 mg/kg of L-2-oxothiazolidine-4-carboxylic acid was orally administered to type 2 diabetes mice for 4 weeks, as shown in EXAMPLE 5.

The lowering effect of blood glucose levels are mostly likely due to the fact that L-2-oxothiazolidine-4-carboxylic acid improves insulin secretion when administered to type 2 diabetes mice.

### EXAMPLE 6

Morphological examination of the OTC effect on insulin secretion

By immunohistochemistry using anti-insulin antibody, the present example is to prove L-2-oxothiazolidine-4-carboxylic acid as an excellent agent for the insulin secretion by comparing the ability of insulin secretion of pancreatic islet cells of the control group as in EXAMPLE 5 and an experimental group having type 2 diabetes to which 80 mg/kg of OTC is administered for four weeks.

L-2-oxothiazolidine-4-carboxylic acid or PBS alone was administered to 3 mice in each group, 1 g/kg of glucose was intraperitoneally injected in the fasted state, and they were sacrificed by cervical dislocation after 120 min. of the injection. Immediately, the abdominal region was incised, pancreases were then removed from the mice and fixed more than 12 hours in a cold solution of 4% paraformaldehyde in PBS(pH 7.4, Sigma, USA). Pancreas separated free of intestine and adipose tissue(fatty tissue), rinsed three times with fresh fixing solution, and the fixed tissues were processed routinely for paraffin embedding. The tissue sections were cut off at 5 ^{μ}m interval using microtome(Sandon, Germany) from the paraffin embedding, and adhered to a slide coated with poly-L-lysine(Sigma, USA) for immunohistochemistry.

To remove the paraffin from the slides on which pancreatic tissue was coated, the slides were repeatedly treated with xylene for 5 min., and the tissue sections were rehydrated with a series of 100-70% alcohol and incubated with PBS(pH 7.4) for 5 minutes. The tissue sections were treated with 3% hydrogen peroxide for the inactivation of endogenous peroxidase, then incubated with anti-insulin antibody(Dako, USA) at 4^{°C.}for 12 hours, and the samples were processed by immunohistochemistry staining method using super-signal amplifying immunohistochemistry kit(A.B.I., Chonju, Korea) in a series of processing. Counterstaining against the tissue samples was performed with hematoxylin, and pictures(200 magnifications) of pancreatic islets were taken after reviewed by 100 magnification-microscope(Leica, Germany). Comparison was made to identify the pancreatic tissues using hematoxylin-eosine(Sigma, USA). The results are shown in FIG. 10. In the hematoxylin-eosine staining morphology, the density of pancreatic -islet cells of the control group to which PBS was given for four weeks was low, and the capsule which surrounded the pancreatic islets was also destroyed(see FIG. 10-a). In contrast, the densities of the capsule and the β-cells in the capsule of the experimental group, to which 80 mg/kg of L-2-oxothiazolidine-4-carboxylic acid was administered for four weeks, were high(see FIG. 10-b).

On the other hand, through the immuno-staining for insulin pancreatic β-islet cells, the positive response to anti-insulin antibody of pancreatic β-islet cells of the control group, to which PBS was given for four weeks was found to be eminently reduced(see FIG. 10-c), in contrast, the positive response to anti-insulin antibody of pancreatic β-islet cells of the experimental group, to which 80 mg/kg of L-2-oxothiazolidine-4-carboxylic acid was administered for four weeks, was distinctly increased(see FIG. 10-d). From the above results, it is clear that L-2-oxothiazolidine-4-carboxylic acid has a beneficial effect on insulin secretion.

The example observed in EXAMPLE 6 proves that the lowering effect of blood glucose levels by the administration of L-2-oxothiazolidine-4-carboxylic acid is most likely due to the fact that the drug has an excellent effect of insulin secretion.

FIG. 10 shows a morphologic examination of pancreatic islets from diabetes mice, taken by 200 magnification microscope. The dark brownish stain indicates the positive response by anti-insulin antibody. (a and b: hematoxylin-eosine staining of pancreatic islets) (c and d: immunohistochemistry of pancreatic islets with anti-insulin antibody) (a and c: the control group to which PBS alone was given for four weeks) (b and d: the experimental group to which 80 mg/kg of L-2-oxothiazolidine-4-carboxylic acid was administered for four weeks) arrow: pancreatic islet.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

We claim:

1. Use of L-2-oxothiazolidine-4-carboxylic acid, its derivatives, or its pharmacologically acceptable salts of the formula(I) 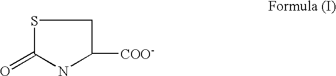
as pharmaceutical active compounds for the treatment of type 2 diabetes.

2. Use of the compounds as defined in claim 1, wherein the salts are selected from the group consisting of sodium, potassium, calcium and magnesium salts of the compound L-2-oxothiazolidine-4-carboxylic acid.

3. Use of the compounds as defined in claim 1, wherein the derivative is ester form of L-2-oxothiazolidine-4-carboxylic acid.

4. Use of the compounds as defined in claim 1, wherein the active compound is administered at the concentration of 3-400 mg/kg.

5. Use of the compounds as defined in claim 1, wherein the active compound is administered at the concentration of 40-80 mg/kg.

6. Use of the compounds as defined in claim 1, wherein they are used for the prevention of diabetes complications.

7. A pharmaceutical composition containing at least one compound of L-2-oxothiazolidine-4-carboxylic acid, its derivatives or pharmacologically acceptable salts of the formula(I) as an active component and a pharmaceutically acceptable excipient, carrier, or diluent.

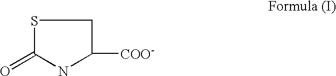

8. The pharmaceutical composition as defined in claim 6, wherein the salts are selected from the group consisting of sodium, potassium, calcium and magnesium salts of the compound L-2-oxothiazolidine-4-carboxylic acid.

9.The pharmaceutical composition as defined in claim 6, wherein the derivative is ester form of L-2-oxothiazolidine-4-carboxylic acid.

10. A method of treating a mammal having a type 2 diabetes, said method comprising administering to said mammal a pharmaceutically effective amount of L-2-oxothiazolidine-4-carboxylic acid, its derivatives, and its pharmacologically acceptable salts of the Formula(I).

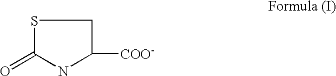

11. The method of treating a mammal having a type 2 diabetes as defined in claim 9, wherein the salts are selected from the group consisting of sodium, potassium, calcium and magnesium salts of the compound L-2-oxothiazolidine-4-carboxylic acid.

12. The method of treating a mammal having a type 2 diabetes as defined in claim 9, wherein the derivative is ester form of L-2-oxothiazolidine-4-carboxylic acid.

13. The method of treating a mammal having a type 2 diabetes as defined in claim 9, wherein the pharmaceutical composition is administered orally.
